(19) United States
(12) Patent Application Publication
(10) Pub. No.: US 2015/0209784 A1
(43) Pub. Date: Jul. 30, 2015

(54) Arrangement for Quantifying Cells of a Cell Suspension

(71) Applicant: SIEMENS AKTIENGESELLSCHAFT, München (DE)

(72) Inventors: Oliver Hayden, Herzogenaurach (DE); Michael Johannes Helou, Regensburg (DE); Lukas Richter, Erlangen (DE)

(21) Appl. No.: 14/412,543

(22) PCT Filed: Jun. 24, 2013
(86) PCT No.: PCT/EP2013/063128

§ 371 Date: Jan. 2, 2015

(30) Foreign Application Priority Data
Jul. 4, 2012 (DE) 102012 211626.5

(51) Int. Cl. B01L 3/00; G01N 15/10; G01N 33/543

(52) U.S. Cl. CPC B01L 3/502761; G01N 33/54326; G01N 15/1031; B01L 2200/0652; B01L 2200/0668; B01L 2300/0816; B01L 2300/0851; B01L 2400/043; B01L 2300/0627

(57) ABSTRACT

The embodiments relate to an arrangement for quantifying cells of a cell suspension and enriching marked cells. The arrangement includes a fluid channel for routing the cell suspension with a first cross-section and a magnetic sensor on the fluid channel for counting magnetically marked cells in the cell suspension. The fluid channel has an enrichment region with a second cross-section which is larger than the first cross-section, a magnet being arranged on at least one side of the enrichment region.

### CROSS-REFERENCE TO RELATED APPLICATIONS

The present patent document is a §371 nationalization of PCT Application Serial Number PCT/EP2013/063128, filed Jun. 24, 2013, designating the United States, which is hereby incorporated by reference, and this patent document also claims the benefit of DE 10 2012 211 626.5, filed on Jul. 4, 2012, which is also hereby incorporated by reference.

### TECHNICAL FIELD

The embodiments relate to an arrangement for quantifying cells of a cell suspension, including a fluid channel for conducting the cell suspension and a magnetic sensor on the fluid channel for counting magnetically labeled cells in the cell suspension.

### BACKGROUND

For the detection of single cells, optical flow cytometry may be used. It provides, using the FACS system (fluorescence activated cell sorting), a way of separating and further using fluorescence-labeled cells of the measured suspension. In this process, the separated cells are, depending on labeling, electrically charged to varying extents and deflected by charged plates into various collection containers.

As an alternative to fluorescence-based detection, magnet-based detection of cells may also be used. For a selective, magnetic detection of single cells, the cells are labeled with superparamagnetic labels and transported across a magnetoresistive component, for example, GMR. The aim is, at a very high cell concentration, to be able to arrange the analytes at a very short distance from one another and detect them individually.

### SUMMARY AND DESCRIPTION

The scope of the present invention is defined solely by the appended claims and is not affected to any degree by the statements within this summary. The present embodiments may obviate one or more of the drawbacks or limitations in the related art.

It is an object of the present embodiments to specify an arrangement and a method that allow a magnet-based detection of single cells and, simultaneously, a separation of magnetically labeled cells from unlabeled cells.

The arrangement for quantifying cells of a cell suspension includes a fluid channel for conducting the cell suspension. Furthermore, the arrangement includes a magnetic sensor on the fluid channel. The sensor may be based on GMR, AMR, or the like, and is designed for counting magnetically labeled cells in the cell suspension. It is useful if the magnetic sensor is located directly next to or within the fluid channel.

The fluid channel includes an enrichment region having an enlarged cross section. A magnet is arranged on at least one side of the enrichment region. The magnet may be an electromagnet. A permanent magnet may be used.

In the method for quantifying cells of a cell suspension and enriching magnetically labeled cells, a fluid channel having a first cross section and having an enrichment region having a second cross section enlarged with respect to the first cross section is provided. The cells in the fluid channel are guided to the enrichment region and to a magnetic sensor there for counting magnetically labeled cells in the cell suspension. The cells in the enrichment region are drawn to one side of the fluid channel by a magnet.

Advantageously, the arrangement achieves not only the pure quantification of the labeled cells, but also a separation of the cells. To this end, the magnet provides an enrichment of the magnetically labeled cells in the enrichment region. Since the non-magnetically labeled cells do not respond to the magnetic field, they are not enriched either and flow unimpeded in the fluid channel away from the magnet. It is useful in this case if the magnetic sensor is arranged in the enrichment region. Thus, it is advantageously possible for not only the measurement of the cells, but also a subsequent separation of the cells and a removal of the magnetically labeled cells to take place. In this case, the magnetically labeled cells are enriched and an act for sorting magnetically labeled cells from unlabeled ones is unnecessary. This approach relieves or spares a complex sample preparation, which is associated with a potential loss of the cell material to be analyzed.

In the arrangement, the magnet may be arranged such that the cells in the enrichment region are drawn from the cross section of the fluid channel that is present beyond the enrichment region. In other words, the cells are drawn from the stream present in the fluid channel. Cells located in the flow-abated parts of the fluid channel in the enrichment region are substantially more easily influenceable by the force effect of the field generated by the magnet and are not so easily carried away by the flow otherwise present in the fluid channel.

It is advantageous when, in the flow direction of the cell suspension at the end of the enrichment region, the fluid channel has a concave shape, designed for capturing magnetically labeled cells drawn into the enrichment region by the magnet. In other words, the fluid channel has, in the region of the expanded cross section toward its end, a pocket or similar shaping that is shaped such that cells, once guided in there, are largely cut off from the stream in the fluid channel and may only get into the fluid channel by moving against the otherwise predominant stream.

### BRIEF DESCRIPTION OF THE DRAWINGS

FIG. 1 depicts an embodiment of an arrangement of a Si-based GMR sensor and of a fluid channel for cell quantification and enrichment.

FIGS. 2-4 depict embodiments of the arrangement in operation.

FIG. 5 depicts an embodiment of process acts when using the arrangement in hematology.

FIG. 6 depicts an embodiment of process acts when using the arrangement in hemostasis analysis.

### DETAILED DESCRIPTION

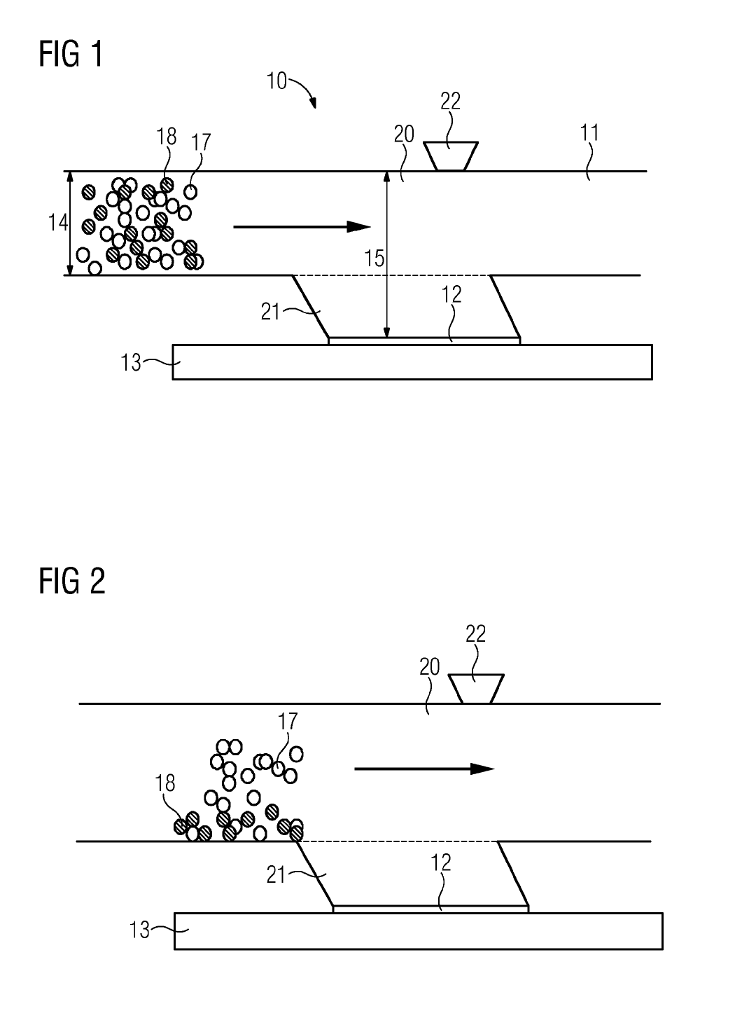

An arrangement 10 for the detection of single cells and subsequent separation of the cells 17, 18 according to FIG. 1 includes a mixing chamber and a fluid channel 11 for enriching the cells and guiding them across a magnetoresistive GMR sensor 12. In the exemplary embodiment according to FIG. 1, the GMR sensor 12 is mounted on a silicon wafer, which is in turn arranged on a permanent magnet 13.

In the arrangement, the fluid channel 11 has, away from the permanent magnet 13, a first cross section 14. In the region of the permanent magnet 13, the fluid channel 11 is broadened and has a second cross section 15 that is greater than the first cross section 14. The increase in the cross section 14 broadens the fluid channel 11 specifically such that the fluid channel 11 reaches up to the silicon wafer having the GMR sensor 12. In the regions having the first cross section 14, the fluid channel 11 is, by contrast, at a distance from the permanent magnet 13.

In the arrangement, the enrichment region 21 arising as a result of the second cross section 15 is formed in the manner of a parallelogram in the view according to FIGS. 1 to 4. As a result, a type of pocket is formed in the flow direction of the cells 17, 18 toward the end of the enrichment region 16. In the present example, the enrichment region 21 is exclusively formed toward the side facing the permanent magnet 13. In the other directions, the fluid channel 11 in the region having a second cross section 15 is unaltered with respect to the region having the first cross section 14.

FIG. 1 depicts a quantity of cells 17, 18. Some of the cells 17 are not magnetically labeled. The rest of the cells 18 are magnetically labeled, for example, using superparamagnetic beads. The labeled and unlabeled cells 17, 18 are mixed together and flow in the fluid channel 11 onto the region having an enlarged cross section 15. To this end, a pump generates a suitable flow in the fluid channel 11.

FIG. 2 depicts the situation at a time at which the cells 17, 18 have almost reached the region having an enlarged cross section 15. The magnetically labeled cells 18 are, under the influence of the permanent magnet 13, initially drawn in the direction of the permanent magnet and concentrated on the corresponding side of the fluid channel 11.

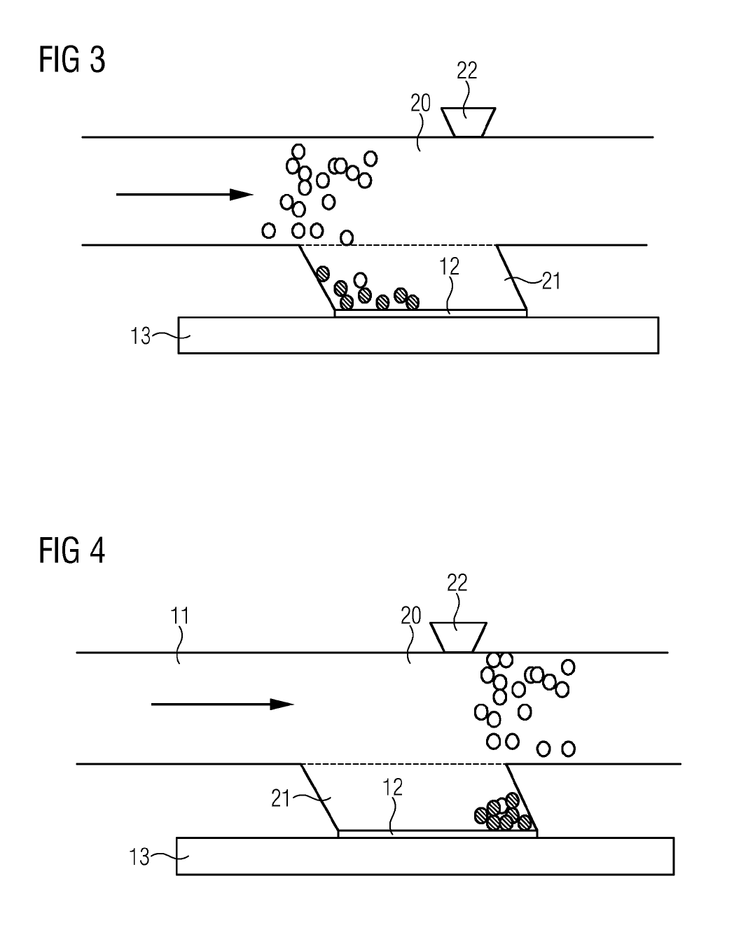

FIG. 3 depicts the situation at a time at which the cells 17, 18 reach the region having an enlarged cross section 15. There they pass through, apart from the interference owing to the permanent magnet 13, the notional continuation 20 of the fluid channel 11 in the region having the second cross section 15. The magnetically labeled cells 18 are, under the influence of the permanent magnet 13, drawn out further from the notional continuation 20 of the fluid channel 11 into the enrichment region 21. In this case, it is also possible that isolated non-labeled cells 17 are drawn along by mutual friction. The majority of the unlabeled cells 17 remains in the notional continuation 20 and is carried away further by the stream in the fluid channel 11.

The magnetically labeled cells 18 are carried by the stream across the GMR sensor 12 and thereby trigger signals, by which it is possible to count the labeled cells.

FIG. 3 depicts the situation at a later time at which the cells 17, 18 reach the end of the region having an enlarged cross section 15. At this time, the labeled cells 18 cluster in the pocket in the enrichment region 21 and may only return from there to the fluid channel 11 by moving against the stream. The labeled cells 18 therefore remain largely in the pocket in the enrichment region 21. The unlabeled cells 17 are, however, carried away by the stream in the fluid channel 11.

The result is that a strong enrichment of the labeled cells 18 in the enrichment region 21 therefore takes place by the described arrangement 10. Nonlabeled cells 17 are flushed away. The labeled cells 18 may then, for example, be removed and be used for performing follow-up analyses. Advantageously, this may achieve a considerable shortening of the work acts for certain test sequences. To this end, the arrangement 10 may, for example, have a septum 22 (e.g., pierceable membrane).

An example of such a test sequence is an analysis of lymphocytopenia, e.g., the excessively low number of lymphocytes. In this case, the arrangement 10 is realized within a point-of-care device. Lymphocytopenia may, for example, occur during the intake of corticoids in the course of an HIV infection (e.g., CD4+ T helper cells), great stress, rheumatoid arthritis or an idiopathic CD4+ lymphocytopenia (e.g., fewer than 300 CD4+ T cells/μl of blood).

FIG. 5 depicts states of the cells 17, 18 that are reached in this example of quantification as a result of certain measurement acts. A first state 501 is reached after the cells 17, 18, both labeled and unlabeled, have been guided in a first flow direction 52 across the GMR sensor 12. In the state, they have already been separated as described for FIGS. 1 to 4 and drawn in the enrichment region 21 toward the permanent magnet 13. In said state, the cells 17, 18 are concentrated at one end of the enrichment region 21.

Then, the flow direction is reversed and the cells 17, 18 therefore flow in a second flow direction 52 toward the other end of the enrichment region 21. While doing so, the cells cross the GMR sensor 12 again, giving rise to the second state 502. While doing so, the cells may be counted once again. In this process, the magnetic guidance structures 51 provide, in the case of the magnetic labeled cells 18, an orientation of the cells 18 toward the center of the enrichment region 21, and so the cells are guided across the GMS sensor increasingly one at a time and in succession. The unlabeled cells 17 do not respond to the magnetic guidance structures 51, and may thus leave the enrichment region 21 again (separated from the labeled cells 18).

The third state 503 arises when, during reversed flow direction, all cells 17, 18 are collected again at the end of the enrichment region 21. Then, the flow direction is reversed again. This gives rise to the fourth state 504, in which the cells 17, 18 (influenced by the magnetic guidance structures 51) pass the GMR sensor 12 again and are counted a third time. Repeated counting allows a statistical evaluation of the results, and thus an increased precision with respect to nonrecurring counting.

Here, the cells to be analyzed 17, 18 may, after the quantification in the arrangement 10, be removed via the septum 22 for further follow-up analysis. An example of such a follow-up analysis is, in relation to HIV infections, for example, the following: in the early stage of an HIV infection, the number of viruses free in blood is very low and may not be identifiable. Infected CD4+ cells, however, may already contain a precursor of HIVs after an infection (e.g., proviruses). In this stage of the infection, any noticeable number of CD4+ cells is not yet measurable (e.g., noticeable: below 500/μl, normal: 600-1600/μl) and the symptoms of the infection are not distinguishable from a conventional influenza. Now, if CD4+ cells are counted, the cells may be subsequently removed and further tested for a possible HIV infection in the early stage.

In a further example, the arrangement 10 is also used as part of a point-of-care device for measuring thrombocytes in order to analyze the process of hemostasis. In this case, the number of thrombocytes is very important especially in conjunction with thrombocytopenia, e.g., an excessively low number of thrombocytes. Known methods capture either only the relative change in thrombocyte number (e.g., cellular branch of hemostasis) or only the plasmatic branch of blood coagulation, e.g., without capturing the number of thrombocytes.

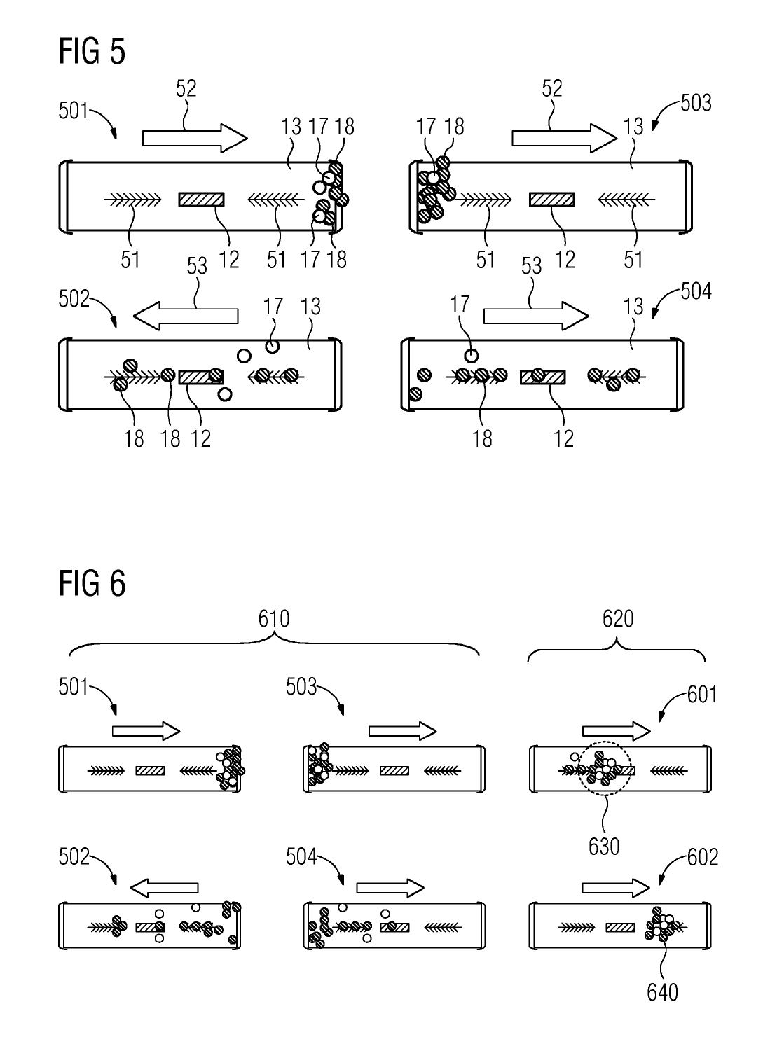

The arrangement 10 described here is capable of measuring both branches of hemostasis (e.g., cellular and plasmatic) by determining the number of thrombocytes at the start of measurement. The acts of measurement and the states arising in this connection are depicted in FIG. 6. In this case, the cells 17, 18 of the cell suspension are initially conducted repeatedly across the sensor region as already described in relation to FIG. 5.

Over the course of time, cell aggregates 630 and lastly a clot containing fibrin 640 are formed. In this case, the number of thrombocytes, their activation and the development of aggregations may be captured using, for example, magnetoresistive methods. The formation of a clot may, for example, be achieved by additional surface-sensitive impedance sensors. In this case, the sample is guided repeatedly across the sensor, with more and more cells being deposited on the surface over the course of time, and this is marked by an increase in impedance.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An arrangement for quantifying cells of a cell suspension and enriching labeled cells, the arrangement comprising:
a fluid channel for conducting the cell suspension having a first cross section; and
a magnetic sensor on the fluid channel for counting magnetically labeled cells in the cell suspension,
wherein the fluid channel comprises an enrichment region having a second cross section enlarged with respect to the first cross section, and
wherein a magnet is arranged on at least one side of the enrichment region.

2. The arrangement as claimed in claim 1, wherein the magnet is arranged such that cells in the enrichment region are drawn from a cross section of the fluid channel present beyond the enrichment region.

3. The arrangement as claimed in claim 1 wherein, in the enrichment region, the second cross section is present in a direction from an axis of the fluid channel.

4. The arrangement as claimed in claim 1, wherein, in a flow direction of the cells at the end of the enrichment region, the fluid channel comprises a concave shape configured to capture magnetically labeled cells drawn into the enrichment region by the magnet.

5. The arrangement as claimed in claim 1, further comprising at least one impedance sensor arranged in a region of the magnetic sensor.

6. The arrangement as claimed in claim 1, further comprising a pierceable membrane.

7. A method for quantifying cells of a cell suspension and enriching magnetically labeled cells, the method comprising:
providing a fluid channel having a first cross section and having an enrichment region having a second cross section enlarged with respect to the first cross section;
guiding cells in the fluid channel to the enrichment region and across a magnetic sensor located next to or within the fluid channel; and
counting magnetically labeled cells in the cell suspension,
wherein the cells in the enrichment region are drawn to one side of the fluid channel by a magnet.

8. The method as claimed in claim 7, further comprising:
reversing a flow direction in the fluid channel after the cells have been guided across the magnetic sensor; and
guiding the cells across the magnetic sensor again.

9. The method as claimed in claim 8, further comprising:
repeating the reversing of the flow direction in the fluid channel and counting the labeled cells when covering the magnetic sensor.

10. The method as claimed in claim 9, further comprising:
carrying out a wash step after the cells have been guided across the magnetic sensor.

11. The method as claimed in claim 8, further comprising:
carrying out a wash step after the cells have been guided across the magnetic sensor.

12. The method as claimed in claim 7, further comprising:
carrying out a wash step after the cells have been guided across the magnetic sensor.

13. The arrangement as claimed in claim 2, wherein, in the enrichment region, the second cross section is present in a direction from an axis of the fluid channel.

14. The arrangement as claimed in claim 13, wherein, in a flow direction of the cells at the end of the enrichment region, the fluid channel comprises a concave shape configured to capture magnetically labeled cells drawn into the enrichment region by the magnet.

15. The arrangement as claimed in claim 14, further comprising at least one impedance sensor arranged in a region of the magnetic sensor.

16. The arrangement as claimed in claim 15, further comprising a pierceable membrane.

17. The arrangement as claimed in claim 3, wherein, in a flow direction of the cells at the end of the enrichment region, the fluid channel comprises a concave shape configured to capture magnetically labeled cells drawn into the enrichment region by the magnet.

18. The arrangement as claimed in claim 5, further comprising a pierceable membrane.

19. The arrangement as claimed in claim 2, further comprising at least one impedance sensor arranged in a region of the magnetic sensor.

20. The arrangement as claimed in claim 2, further comprising a pierceable membrane.
